(19) United States
(12) Patent Application Publication
(10) Pub. No.: US 2007/0185525 A1
(43) Pub. Date: Aug. 9, 2007

(54) Floating on the wire filter wire

(76) Inventors: Bradley R. White, Palo Alto, CA (US); Rajan D. Khokhar, Santa Cruz, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/349,330

(22) Filed: Feb. 7, 2006

(51) Int. Cl. A61M 29/00

(52) U.S. Cl. 606/200

(57) ABSTRACT

Embolic protection filtering devices and methods for making and using the same. An example filtering device includes a filter wire, a filter slidable over the filter wire, and a filter membrane coupled to the filter frame. The filter wire includes a plurality of stops. The filter includes a proximal tubular member, a distal tubular member, and a connecting tubular member extending between the proximal tubular member and the distal tubular member.

### FIELD OF THE INVENTION

The present invention pertains to embolic protection filtering devices. More particularly, the present invention pertains to embolic protection filtering device with position-stabilizing features and characteristics.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of filtering devices have been developed for medical use, for example, intravascular use. Of the known filtering devices, each has certain advantages and disadvantages. There is an ongoing need to provide alternative filtering devices as well as alternative methods for manufacturing filtering devices.

### BRIEF SUMMARY

This disclosure pertains to design, material, and manufacturing method alternatives for filtering devices. An example filtering device includes a filter wire, a filter slidable over the filter wire, and a filter membrane coupled to the filter. The filter wire includes a plurality of stops. The filter includes a proximal tubular member, a distal tubular member, and a connecting tubular member extending between the proximal tubular member and the distal tubular member.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is partial cross-sectional side view of an example filtering device disposed in a blood vessel;

FIG. 2 is a side view of the example filtering device shown in FIG. 1 where the filter is in a first position relative to the filter wire; and

FIG. 3 is a side view of the example filtering device shown in FIGS. 1-2 where the filter is in a second position relative to the filter wire.

### DETAILED DESCRIPTION

The following description should be read with reference to the drawings wherein like reference numerals indicate like elements throughout the several views. The detailed description and drawings illustrate example embodiments of the claimed invention.

When a clinician performs an intravascular intervention such as angioplasty, atherectomy, and the like, embolic debris may dislodge from the blood vessel that can travel in the bloodstream to a position where it may impair blood flow, possibly leading to tissue damage. A number of other situations and/or interventions may also result in the mobilization of embolic debris. Accordingly, embolic protection filtering devices have been developed that can be disposed in the blood vessel downstream of the treatment site and expanded to capture debris.

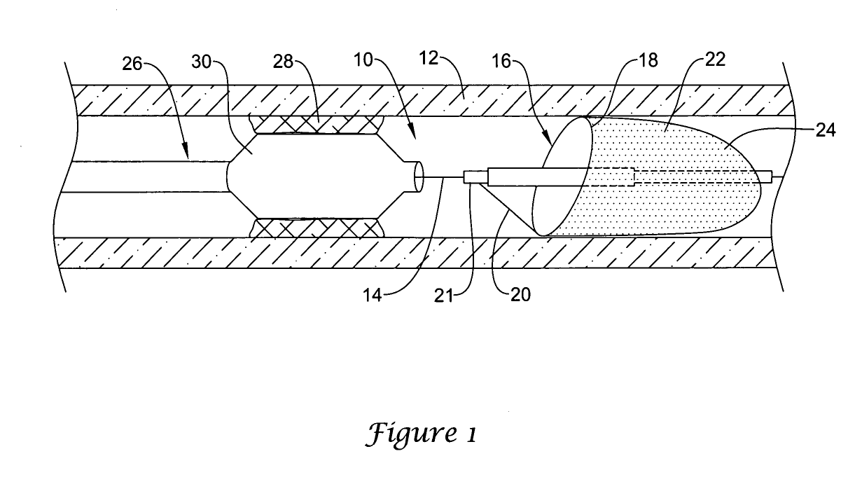

FIG. 1 is a partial cross-sectional view of an example embolic protection filtering device 10 disposed within a blood vessel 12. Device 10 may include an elongate shaft or filter wire 14 having an embolic protection filter 16 coupled thereto. Filter 16 includes a filter loop 18 and a filter membrane or fabric 22 coupled to filter loop 18. Filter membrane 22 can be drilled (for example, formed by known laser techniques) or otherwise manufactured to include a plurality of openings 24. These holes or openings 24 can be sized to allow blood flow therethrough but restrict flow of debris or emboli floating in the body lumen or cavity.

In general, filter 16 may be adapted to operate between a first generally collapsed configuration and a second generally expanded configuration for collecting debris in a body lumen. To this end, in at least some embodiments, loop 18 may be comprised of a “self-expanding” shape-memory material such as nickel-titanium alloy, which is capable of biasing filter 16 toward being in the second expanded configuration. Additionally, filter loop 18 may include a radiopaque material or include, for example, a radiopaque wire disposed about a portion thereof. Some further details regarding these and other suitable materials are provided below.

One or more struts 20 may extend between filter loop 18 and filter wire 14. Strut 20 may be coupled to filter wire 14 by a coupling 21. Coupling 21 may be one or more windings of strut 20 about filter wire 14 or may be a fitting disposed over an end of strut 20 to attach it to filter wire 14. The exact arrangement of struts 20 can vary considerably. One of ordinary skill in the art would be familiar with the various arrangements of struts 20 that are appropriate for a given intervention.

With filter 16 properly positioned in blood vessel 12, another medical device may be advanced over filter wire 14 in order to treat and/or diagnose a lesion 28. For example, a catheter 26 (such as the balloon catheter depicted in FIG. 1) may be advanced over filter wire 14 in order to expand lesion 28. Of course numerous other devices could just as easily be passed over filter wire 14 including any device designed to pass through an opening or body lumen. For example, the device may comprise any type of catheter (e.g., therapeutic, diagnostic, or guide catheter), a stent delivery catheter, an endoscopic device, a laproscopic device, variations and refinements thereof, and the like, or any other suitable device. Alternatively, another device may be advanced over or through its own guiding structure to a suitable location adjacent filter 16 in a manner that allows device 10 to perform its intended filtering function.

Filtering device 10 is generally designed to filter embolic debris that might be generated during the course of this medical intervention. For example, device 10 can be used to capture embolic debris that might be generated during the use of catheter 26 such as when a balloon 30 (coupled to catheter 26) is inflated. It should be noted, however, that device 10 may find utility in concert with essentially any procedure that has the potential to loosen and release embolic debris in to the blood stream or with the devices associated with such procedures.

Maintaining the position of a filtering device within a blood vessel during an intervention may be desirable. For example, if the filter migrates within the vessel during an intervention, the filter could come into contact with another device (e.g., a catheter disposed on filter wire 14) and potentially interfere with the goals of the intervention. In addition, advancing other devices over the filter wire may cause small shifts in the position of the filter wire itself that may take the filter out of its optimal position. Additionally, at some points during an intervention, it may be desirable for movements (small or large) to be directly translated onto the filter so that the filter can be placed in the proper position. In at least some embodiments, the present invention addresses these and other needs by providing structural features that allow filter 16 to either be “fixed” relative to filter wire 14 (so that any movement of filter wire 14 directly translates to analogous movement of filter 16) or be “position-stabilized” so that relatively small movements of filter wire 14 do not result in movement of filter 16. It should be noted that the term “fixed” is understood to mean that filter 16 is coupled to filter wire 14 so that any movement of filter wire 14 directly translates to analogous movement of filter 16. It should be noted that the term “fixed” is not meant to imply that in all embodiments of filtering device 10, filter 16 may become permanently attached and secured to filter wire 14. At least some embodiments of filtering device 10 are contemplated where filter 16 can shift out of the “fixed” configuration and into another configuration.

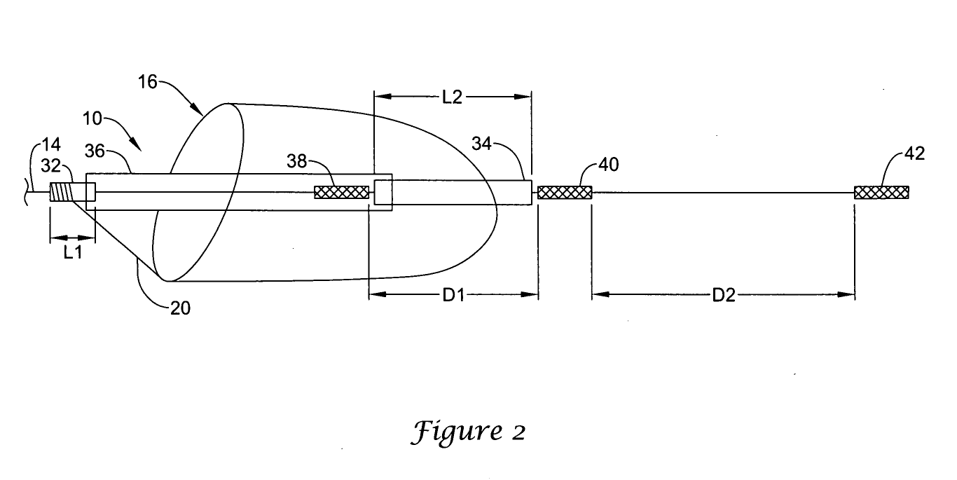

Turning now to FIG. 2, here it can be seen that filter 16 includes a first or proximal tubular member 32, a second or distal tubular member 34, and a connecting tubular member 36 coupled to and extending between proximal tubular member 32 and distal tubular member 34. Proximal tubular member 32 has a length L1 and distal tubular member has a length L2.

Filter wire 14 includes a plurality of stops, for example first stop 38, second stop 40, and third stop 42. First stop 38 and second stop 40 are separated from each other by a distance D1. Second stop 40 and third stop 42 are separated from each other by a distance D2. Filter wire 14 may take the general form of typical filter wires in the art. Stops 38/40 are generally configured to allow filter 16 (i.e., tubular members 32/34) to more easily slide thereover in the distal direction but be more difficult to pass in the proximal direction. Stop 42, in at least some embodiments, may be disposed at the distal end of filter wire 14 and define the distal tip of filter wire 14. Alternatively, stop 42 may be set back a distance from a spring tip or solder ball tip.

The arrangement and configuration of the various components listed above is designed so that filter 16 can be slid over filter wire 14 to one or more positions where filter 16 can either be “fixed” relative to filter wire 14 (so that any movement of filter wire 14 directly translates to analogous movement of filter 16) or be “position-stabilized” so that relatively small movements of filter wire 14 do not result in movement of filter 16. In at least some embodiments, these features are attributed to filtering device 10 by virtue of the sizing of tubular members 32/34 (e.g., their respective lengths L1/L2) and the distances D1/D2 between stops 38/40/42. In general, the length L2 of distal tubular member 34 is substantially the same as distance D1 between first stop 38 and second stop 40. Because of this, if distal tubular member 34 is disposed between first stop 38 and second stop 40, filter 16 is fixed or locked on filter wire 14 so that any movement of filter wire 14 directly translates to analogous movement of filter 16.

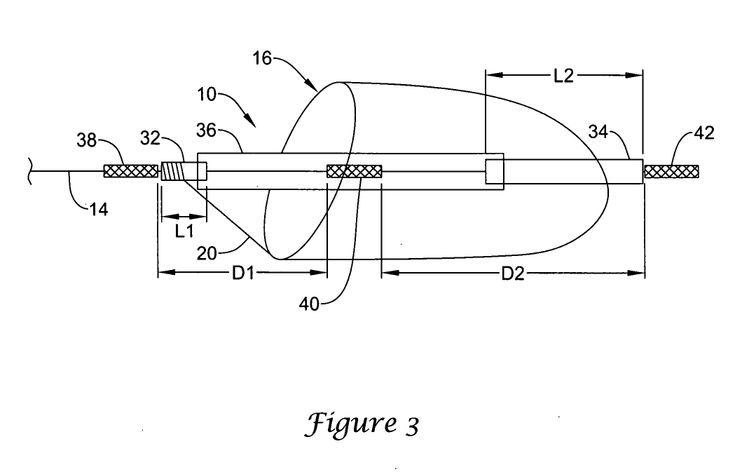

The length L2 of distal tubular member 34, in contrast, is shorter than distance D2 between second stop 40 and third stop 42. In addition, the length L1 of proximal tubular member 32 is shorter than distance D1 between first stop 38 and second stop 40. Because of this configuration, if distal tubular member 34 is disposed between second stop 40 and third stop 42 and if proximal tubular member 32 is disposed between first stop 38 and second stop 40 (as shown in FIG. 3), filter 16 is “position-stabilized” so that relatively small movements of filter wire 14 do not result in movement of filter 16.

Connecting tubular member 36 is generally configured to fit over and connect proximal tubular member 32 with distal tubular member 34. At least some embodiments of connecting tubular member 36 are configured to slidably pass over stops 38/40 so that the arrangements described above can be realized. Otherwise, connecting tubular member 36 can be configured in any suitable manner.

It should be noted that a number of different embodiments are contemplated where the precise size arrangement or configuration differs from what is described above. For example, some embodiments of filtering device 10 include distal tubular member 34 with a length L2 that is shorter than distance D1. Because of this, if distal tubular member 34 is disposed between first stop 38 and second stop 40, filter 16 is “position-stabilized” so that relatively small movements of filter wire 14 do not result in movement of filter 16. In some of these embodiments, length L2 of distal tubular member 34 can be closer in length to one or either distance D1 or distance D2. In this arrangement, filter 16 can take either of two different “position-stabilized” positions. In one of the positions (e.g., with distal tubular member 34 disposed between second stop 40 and third stop 42 and where length L2 is closer in size to D1 than to D2), filter 16 may be able to hold its position when exposed to a greater amount of movement of filter wire 14 than other positions. For example, in another position, (e.g., with distal tubular member 34 disposed between first stop 38 and second stop 40 and where length L2 is closer in size to D1 than to D2), filter 16 may be able to hold its position when exposed to a lesser amount of movement of filter wire 14 than the other position. Thus, this arrangement allows the clinician to choose between two different filter 16 positions that each has a different amount of “play” (i.e., different levels of position-stabilization). Other arrangements are contemplated including the reverse of what is described above.

The precise dimensions for lengths L1/L2 and distances D1/D2 can vary. For example, in some embodiments, lengths L1/L2 can each be about 0.1-5 inches, 0.1-2 inches, or about 0.1-1 inch or so. Similarly, D1/D2 can each be about 0.1-5 inches, 0.1-2 inches, or about 0.1-1 inch or so. It can be appreciated that these dimensions are not intended to limit the invention to any particular size as embodiments are contemplated that include dimensions within as well as outside the above ranges. In addition, FIG. 2 illustrates a small amount of space between distal tubular member 34 and first stop 32 and second stop 34 for illustration purposes. It can be appreciated that the precise amount of spacing that exists is appropriate for achieving the desired effect.

A number of methods are contemplated for using filtering device 10. For example, a method for filtering embolic debris in a body lumen includes the steps of providing filtering device 10, advancing filter wire 14 through a body lumen to a position adjacent an area of interest, advancing filter 16 over filter wire 14 to a first position where distal tubular member 34 is disposed between first stop 38 and second stop 40, further advancing filter 34 over filter wire 14 to a second position where proximal tubular member 32 is disposed between first stop 38 and second stop 40, and deploying filter 16. Depending on the arrangement of the various elements of filtering device 10, filter 16 may be fixed relative to filter wire 14 when in the first position and position-stabilized when in the second position. In some embodiments, the step of advancing filter 16 over filter wire 14 may include the use of a suitable delivery catheter.

The overall design of filtering device 10 includes the use of a number of different materials appropriate for the various components thereof. These materials may include metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®, ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID(® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In addition, filtering device 10 or portions thereof, may also be doped with or otherwise include a radiopaque material as stated above in relation to filter loop 18. Radiopaque materials are understood to be materials capable of producing a relatively bright image on a fluoroscopy screen or another imaging technique during a medical procedure. This relatively bright image aids the user of filtering device in determining their location. Some examples of radiopaque materials can include, but are not limited to, gold, platinum, molybdenum, palladium, tantalum, tungsten or tungsten alloy, plastic material loaded with a radiopaque filler, and the like.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An embolic protection filter, comprising:
an elongate filter wire;
a proximal stop coupled to the filter wire;
a distal stop coupled to the filter wire and disposed distally of the proximal stop a first distance;
a filter coupled to the filter wire, the filter comprising:
a filter loop,
a proximal tubular member,
a strut extending between the filter loop and the proximal tubular member,
a filter membrane coupled to the filter loop and extending distally therefrom,
a distal tubular member coupled to the filter, and
a connecting tube disposed over the filter wire and connected to both the distal tubular member and the proximal tubular member; and
wherein the proximal tubular member has a length and wherein the length of the proximal tubular member is shorter than the first distance between the proximal stop and the distal stop so that when the proximal tubular member is disposed between the proximal tubular member and the distal tubular member the filter wire can be moved independently of the filter.

2. The filtering device of claim 1, wherein the distal tubular member has a length and wherein the length of the distal tubular member is substantially equal to the first distance between the proximal stop and the distal stop.

3. The filtering device of claim 2, wherein the filter is locked onto the filter wire when the distal tubular member is disposed between the proximal stop and the distal stop.

4. The filtering device of claim 1, further comprising a third stop coupled to the filter wire and disposed distally of the distal stop.

5. The filtering device of claim 4, wherein the third stop is disposed distally of the distal stop a second distance.

6. The filtering device of claim 5, wherein the distal tubular member has a length and wherein the length of the distal tubular member is shorter than the second distance between the third stop and the distal stop so that when the distal tubular member is disposed between the distal stop and the third stop the filter wire can be moved independently of the filter.

7. The filtering device of claim 6, wherein the proximal tubular member is disposed between the distal stop and the proximal stop when the distal tubular member is disposed between the distal stop and the third stop.

8. An embolic protection filtering device, comprising:
a first tubular member having a first length;
a second tubular member having a second length;
a connecting tubular member, the connecting tubular member having a first end coupled to the first tubular member, and a second end coupled to the second tubular member;
a filter disposed adjacent the connecting tubular member, the filter having a strut region coupled to the first tubular member and a filter region coupled to the second tubular member;
a filter wire slidably extending through both the first tubular member and the second tubular member;
a first stop coupled to the filter wire;
a second stop coupled to the filter wire and disposed distally a first distance from the first stop; and
wherein the first length is shorter than the first distance so that when the first tubular member is disposed between the first stop and the second stop the filter wire can be moved independently of the filter.

9. The filtering device of claim 8, wherein the second tubular member has a length and wherein the length of the second tubular member is substantially equal to the first distance between the first stop and the second stop.

10. The filtering device of claim 9, wherein the filter is locked onto the filter wire when the second tubular member is disposed between the first stop and the second stop.

11. The filtering device of claim 8, further comprising a third stop coupled to the filter wire and disposed distally of the second stop.

12. The filtering device of claim 11, wherein the third stop is disposed distally of the second stop a second distance.

13. The filtering device of claim 12, wherein the second tubular member has a length and wherein the length of the second tubular member is shorter than the second distance between the third stop and the second stop so that when the second tubular member is disposed between the second stop and the third stop the filter wire can be moved independently of the filter.

14. The filtering device of claim 13, wherein the first tubular member is disposed between the first stop and the second stop when the second tubular member is disposed between the second stop and the third stop.

15. A method for filtering embolic debris in a body lumen, comprising the steps of:
providing an embolic protection filtering device, the device comprising:
a first tubular member having a first length,
a second tubular member having a second length,
a connecting tubular member, the connecting tubular member having a first end coupled to the first tubular member, and a second end coupled to the second tubular member,
a filter disposed adjacent the connecting tubular member, the filter having a strut region coupled to the first tubular member and a filter region coupled to the second tubular member,
a filter wire slidably extending through both the first tubular member and the second tubular member,
a first stop coupled to the filter wire, and
a second stop coupled to the filter wire and disposed distally a first distance from the first stop, wherein the first length is shorter than the first distance;
advancing the filter wire through a body lumen to a position adjacent an area of interest;
advancing the filter over the filter wire to a first position where the second tubular member is disposed between the first stop and the second stop;
further advancing the filter over the filter wire to a second position where the first tubular member is disposed between the first stop and the second stop; and
deploying the filter.

16. The method of claim 15, wherein movement of the filter wire results in analogous movement of the filter when the filter is in the first position.

17. The method of claim 15, wherein the filter wire can be moved independently of the filter when the filter is in the second position.

18. The method of claim 15, further comprising the step of collapsing the filter.

19. The method of claim 15, further comprising the step of retracting the filter wire.

20. The method of claim 19, wherein the step of retracting the filter wire place the filter in the first position.

21. A filtering device, comprising:
an elongate filter wire having a first stop, a second stop disposed distally a first distance from the first stop, and a third stop disposed distally a second distance from the second stop; and
a filter slidably disposed on the filter wire, the filter including a filter loop, a proximal tubular member having a length shorter than the first distance, a strut extending between the filter loop and the proximal tubular member, a filter membrane coupled to the filter loop and extending distally therefrom, a distal tubular member coupled to the filter, and a connecting tube disposed over the filter wire and connected to both the distal tubular member and the proximal tubular member.

22. The filtering device of claim 21, wherein the distal tubular member has a length that is substantially equal to the first distance.

23. The filtering device of claim 22, wherein the distal tubular member has a length that is shorter than the second distance.
